(19) United States
(12) Patent Application Publication
(10) Pub. No.: US 2026/0038677 A1
(43) Pub. Date: Feb. 5, 2026

(54) NEUROSTIMULATION PARAMETER AUTHENTICATION AND EXPIRATION SYSTEM FOR NEUROSTIMULATION

(71) Applicant: SetPoint Medical Corporation, Valencia, CA (US)

(72) Inventors: Guillermo Alonso CALLE, Moorpark, CA (US); Jacob Aaron JOHNSTON, Moorpark, CA (US)

(21) Appl. No.: 19/356,022

(22) Filed: Oct. 10, 2025

Related U.S. Application Data
(63) Continuation of application No. 18/562,283, now Pat. No. 12,444,497.
(60) Provisional application No. 63/189,638, filed on May 17, 2021.

(51) Int. Cl. G16H 40/40; G16H 20/30; G16H 40/67; H04L 9/32

(52) U.S. Cl. CPC G16H 40/40; G16H 20/30; G16H 40/67; H04L 9/3247; H04L 9/3268

(57) ABSTRACT

Methods and apparatuses (e.g., devices and systems) for securely accessing, activating and/or updating vagus nerve stimulators. In particular, described herein are methods and apparatuses for securing providing, verifying and/or executing digital prescriptions for implantable (including implanted) vagus nerve stimulation systems that may be used for treating inflammation. These methods and apparatuses (including software and/or firmware) may be adapted for used with low-energy requirements of the implanted components.

### CLAIM OF PRIORITY

This patent application is a continuation of U.S. patent application Ser. No. 18/562,283, titled “NEUROSTIMULATION PARAMETER AUTHENTICATION AND EXPIRATION SYSTEM FOR NEUROSTIMULATION,” filed on Nov. 17, 2023, now U.S. Pat. No. 12,444,497, which is a U.S. national phase application under 35 U.S.C. 371 of International Patent Application No. PCT/US2022/029706, titled “NEUROSTIMULATION PARAMETER AUTHENTICATION AND EXPIRATION SYSTEM FOR NEUROSTIMULATION,” filed on May 17, 2022, now International Patent Publication No. WO2022/245878, which claims priority to U.S. Provisional Patent Application No. 63/189,638, filed on May 17, 2021 and titled “NEUROSTIMULATION PARAMETER AUTHENTICATION AND EXPIRATION SYSTEM FOR NEUROSTIMULATION,” each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Examples of the invention relate generally to apparatuses (e.g., systems and devices) and methods of establishing neuronal stimulation. In particular, described herein are implantable microstimulation (MS) devices for treatment of chronic inflammation adapted for electrically stimulating one or more nerves (e.g., the vagus nerve) to treat chronic inflammation by modulation of the inflammatory response via the nicotinic cholinergic anti-inflammatory (“NCAP”) pathway. These apparatuses may be inductively charged. Any of the methods and apparatuses described herein may be in particular adapted for stimulation of the vagus nerve below the diaphragm (e.g., sub-diaphragmatic vagus nerve stimulation).

### BACKGROUND

Electrical stimulation of the cholinergic anti-inflammatory pathway (NCAP) by stimulation of the carotid vagus nerve been well described. For example, see U.S. Pat. Nos. 6,838,471, 8,914,114, 9,211,409, 6,610,713, 8,412,338, 8,996,116, 8,612,002, 9,162,064, 8,855,767, 8,886,339, 9,174,041, 8,788,034 and 9,211,410, each of which is herein incorporated by reference in its entirety.

Implantable electrical stimulation devices have been developed for therapeutic treatment of a wide variety of diseases and disorders. For example, implantable cardioverter defibrillators (ICDs) have been used in the treatment of various cardiac conditions. Spinal cord stimulators (SCS), or dorsal column stimulators (DCS), have been used in the treatment of chronic pain disorders including failed back syndrome, complex regional pain syndrome, and peripheral neuropathy. Peripheral nerve stimulation (PNS) systems have been used in the treatment of chronic pain syndromes and other diseases and disorders. Functional electrical stimulation (FES) systems have been used to restore some functionality to otherwise paralyzed extremities in spinal cord injury patients.

Typical implantable electrical stimulation systems may include one or more programmable electrodes on a lead that are connected to an implantable pulse generator (IPG) that contains a power source and stimulation circuitry. However, these systems can be difficult and/or time consuming to implant, as the electrodes and the IPG are usually implanted in separate areas and therefore the lead must be tunneled through body tissue to connect the IPG to the electrodes. Also, leads are susceptible to mechanical damage over time, particularly as they are usually thin and long.

Recently, small implantable neural stimulator technology, i.e. microstimulators, having integral electrodes attached to the body of a stimulator has been developed to address the disadvantages described above. This technology allows the typical IPG, lead and electrodes described above to be replaced with a single integral device. Integration of the lead has several advantages including reduction of surgery time by eliminating, for example, the need for implanting the electrodes and IPG in separate places, the need for a device pocket, the need for tunneling to the electrode site, and requirements for strain relief ties on the lead itself. Reliability may therefore be increased significantly, especially in soft tissue and across joints because active components, such as lead wires, are now part of the rigid structure and are not subject to the mechanical damage due to repeated bending or flexing over time.

There remains a need for a leadless integral device that is stably positioned on the nerve, and can provide for removal and/or replacement of the stimulation device with relative ease.

Charging and/or communication with an implant by electrical induction (e.g., via one or more inductive coils) may be well suited for use with implantable microstimulators, including those adapted for use to treat inflammation. However, induction may be difficult, particularly where the implant is located deep within the body, as may be the case with a sub-diaphragmatic implant, or where the orientation is not known or is difficult to align with. In previous iterations of the recharging portion of the system, the recharger included a coil that could be worn around a patient's neck. In this configuration, the coil is able to generate an electromagnetic field having sufficient strength to penetrate the patient's body and reach the implanted device for recharging the implanted device. While this recharging scheme is effective, it requires the patient to periodically wear a ring around their necks.

Described herein are microstimulators, charging systems, and methods of using them that may address some of the needs identified above.

Although stimulation of the vagus nerve at the upper levels has been well characterized, stimulation of the NCAP pathway at more distal sites, including sub-diaphragmatic sites has not been well characterized, and poses unique problems and opportunities.

For example, stimulation of sub-diaphragmatic sites may provide fewer adverse events and particularly possibly providing fewer undesirable cardiac effects and laryngeal effects. However, sub-diaphragmatic placement has not been characterized, and may be expected to have a lower efficacy. In addition, the NCAP pathways in sub-diaphragmatic regions may be difficult to access and provide stable placement of a microstimulator.

Also described herein are methods and apparatuses that may address the issues raised above.

In general, the methods described herein may methods and system (including systems for performing any of these methods) for verifying information transmitted to an implanted therapy system (e.g., an implanted neurostimulator). The information transmitted may, in particular, include a clinician's settings for the neuromodulation and/or for a number of therapeutic treatment (e.g., stimulation treatments), and/or verifying parameters related to therapeutic treatments, including but not limited to the date and/or time.

For example, described herein are methods of verifying a clinician authorization that may include verifying one or more digital certificates, e.g., by verifying a digital signature. For example a system may be configured as a system for authorizing communication with an implantable therapy system in which a clinical user is authenticated by the cloud (e.g., a remote server) using a username and password. The cloud server may then issue a digitally signed certificate (that indicates the user is a clinician) with either an implant-specific unique key known only to the implant and the cloud server, or a private key known only to the cloud server, and a public key preprogrammed on all implants. The implantable system may verify the digital signature on the certificate before allowing communication to the implant device to proceed.

Any of the methods and systems described herein may also ensure the accuracy and/or authenticity of the date and/or time updates. For example, these methods and systems may be configured to verify the setting of a date and/or time on an implantable therapy system in which the date and/or time is digitally signed with either an implant-specific unique key known only to the implant and the cloud server, or a private key known only to the cloud server and a public key preprogrammed on all implants. The implant may verify the digital signature on the date and time before allowing its time and/or date to be updated.

Any of these devices and methods may include ensuring authenticity of firmware updates. For example a method and system of updating software and/or firmware deployed on an implantable therapy system may include verifying firmware data that is digitally signed with either an implant-specific unique key known only to the implant and the cloud server or a private key known only to the cloud server, and a public key preprogrammed on all implants. The implant may verify the digital signature on the firmware before the firmware becomes active.

The methods and systems described herein may also or alternatively include ensuring authenticity and clinician authorization of stimulation parameter updates with digital certificates. For example, a system of setting parameters on an implantable therapy device may be configured to authenticate, in a cloud server, a clinical user using a username and password. The cloud server may issue a digitally signed certificate (that signs the parameter data) with either an implant-specific unique key known only to the implant and the cloud server or a private key known only to the cloud server, and a public key preprogrammed on all implants. The cloud server may digitally sign the parameters update information. The implant device may verify the digital signature on the parameters before allowing the parameters to be applied.

Any of these device and methods may be configured to enforce the expiration date on stimulation parameters (e.g., the expiration date of a “prescribed” stimulation treatment). For example, described herein are systems of setting of delivering therapy on an implantable therapy device in which the parameters contain an expiration date and/or time, and the date and time may be enforced to be valid per the mechanism as described above, and the parameters (including the expiration date) may be enforced to be valid per the mechanism described above. In some examples, the therapy may be suspended or prevented from occurring should the expiration date and time be earlier than the current date and time according to the implant's records.

Any of these methods and apparatuses (e.g., systems) may include using digital dose counts as a fallback for an inaccurate clock. For example, any of these methods may be used even where the accuracy of the implantable device's date and time is not recognized to be correct. A fallback mechanism of counting “digital doses” (i.e., periods of stimulation occurring) may be tracked and counted in nonvolatile memory, and if the “digital dose” count is exceeded, therapy may be suspended or prevented from occurring should the expiration date and time be earlier than the current date and time according to the implant's records.

These methods and systems may include allowing clinicians to remotely update stimulation parameters and verifying their authenticity. For example, described herein are systems of setting of delivering therapy on an implantable therapy device in which a clinical user is authenticated by the cloud using a username and password and the clinical user issues a parameter update to one or more patient's implantable devices; the cloud server may issue a digitally signed certificate (that signs the parameter data) with either an implant-specific unique key known only to the implant and the cloud server, or a private key known only to the cloud server, and a public key preprogrammed on all implants. The digitally signed parameter update may be transmitted to the patient's device via the Internet (including any intermediary devices, such as the patient's mobile phone). The implant device may verify the digital signature on the parameters before allowing the parameters to be applied.

Any of these methods and systems may include allowing clinicians to renew expired prescriptions. For example, the methods and system described herein, including those that verify and allow updating of the expiration time, and/or that verify and allow updated “digital dose” count may include notifying patients of expired prescriptions in a patient-facing application (e.g., an app or other software). In some cases, the methods or system may include indicating to a patient that their implanted device's therapy will soon expire on their desktop, mobile device, tablet, wearable, etc.

These methods and apparatuses (including the patient-facing user interface/software) may be configured to allow patients to request prescription refills. In some examples, the methods and system(s) may allow a patient to request a renewal of their implanted therapy from their desktop, mobile device, tablet, wearable, etc. The method and systems may further allow the doctor or clinician to reject the request for prescription refills and assist in enforcing office visit. For example, these methods and apparatuses may allow a clinician to reject the request and, instead, may notify the patient (on their desktop, mobile device, tablet, wearable, etc.) that they need to visit their physician.

For example, a method of authorizing communication with an implanted therapy system may include: receiving, in the implanted therapy system, a communication from a user with a digitally signed certificate, wherein the digitally signed certificate was provided to the user by a remote server after verifying that the user is a clinician who has provided a recognized user name and password to the remote server; verifying, by the implanted therapy system, that a digital signature on the digitally signed certificate corresponds to an authorized user by either: using a unique, implant-specific key to confirm the digital signature, wherein the implant-specific key is known only to the implanted therapy system and to the remote server, or using a public key maintained in the implanted therapy system to confirm the digital signature corresponds to a private key held by only the remote server; and modifying operation of the implanted therapy system based on the communication received from the authorized user.

The modifying operation of the implanted therapy system may comprise modifying a date and/or a time setting for the implantable therapy system. In some cases, the modifying operation of the implanted therapy system may comprise setting electrical stimulation parameters for the implantable therapy system. For example, the electrical stimulation parameters may include an expiration date for the electrical stimulation parameters.

Any of these methods (or systems for performing them) may also include transmitting, from the implanted therapy system to a user-accessible external processor, a notification that a predetermined number of stimulation treatments have been delivered or a predetermined date and/or time will be exceeded within a threshold number of stimulation treatments and/or a threshold period of time. For example, the methods (or systems for performing them) may include displaying the notification in a user interface of the user-accessible external processor. Any of these methods and systems may include facilitating the user requesting additional stimulation treatments on the user interface of the user-accessible external processor. For example, these methods may include denying the user additional stimulation treatments and prompting the user to visit a physician on the user interface of the user-accessible external processor.

Any of these methods may include suspending electrical stimulation by the implanted therapy system after the expiration date or after a specified number of stimulation treatments have been delivered as counted in a nonvolatile memory of the implanted therapy system. For example, suspending electrical stimulation by the implanted therapy system after the expiration date.

As mentioned, these methods (or systems for performing them) may include verifying the date and/or time of the implanted therapy system, e.g., by: receiving, in the implanted therapy system, a date and/or time setting for the implanted therapy system with a second digitally signed certificate; verifying, by the implanted therapy system, that a digital signature on the second digitally signed certificate corresponds to an authorized date and/or time setting by either: using a unique, implant-specific key to confirm the digital signature, wherein the implant-specific key is known only to the implanted therapy system and to the remote server, or using a public key maintained in the implanted therapy system to confirm the digital signature corresponds to a private key held by only the remote server; and modifying the date and/or time setting of the implanted therapy system based on the received date and/or time setting once the digital signature on the second digitally signed certificate is verified.

For example, these methods (or systems for performing them) may include suspending electrical stimulation by the implanted therapy system after the expiration date if the time/date of the implanted therapy system has been confirmed as correct.

Any of these methods and systems may include renewing the expiration date and/or time by receiving a second communication from the user that is verified by the implanted therapy system by verifying a digital signature on a second digitally signed certificate received with the second communication including the renewed expiration date and/or time.

In general, the electrical stimulation parameters may include a specified number of stimulation treatments (e.g., “doses”), which may be monitored by the implanted/implantable system, including by a memory in the implanted/implantable system.

Thus, any of the methods and systems described herein may be configured to suspend electrical stimulation by the implanted therapy system after the specified number of stimulation treatments has been delivered as counted in a nonvolatile memory of the implanted therapy system.

The communication from the user with a digitally signed certificate may be received by the implanted therapy system from the internet and/or a patient's smartphone. In some examples modifying operation of the implanted therapy system may include updating the software and/or firmware based on the communication.

Thus, described herein are methods and systems for verifying and updating the implant, including updating/correcting/verifying the date and/or time, or updating the firmware/software, etc.

For example, a method of setting parameters of an implanted therapy system may include: receiving, in the implanted therapy system, a set of neural stimulation parameters from a user with a digitally signed certificate, wherein the digitally signed certificate was provided to the user by a remote server after verifying that the user is a clinician who has provided a recognized user name and password to the remote server; verifying, by the implanted therapy system, that a digital signature on the digitally signed certificate corresponds to an authorized user by either: using a unique, implant-specific key to confirm the digital signature, wherein the implant-specific key is known only to the implanted therapy system and to the remote server, or using a public key maintained in the implanted therapy system to confirm the digital signature corresponds to a private key held by only the remote server; and setting neural stimulation parameters of the implanted therapy system based on the set of neural stimulation parameters received from the authorized user.

A method of setting a date and/or time of an implanted therapy system may include: receiving, in the implanted therapy system, a date and/or time setting for the implanted therapy system with a digitally signed certificate, wherein the digitally signed certificate was provided by a remote server; verifying, by the implanted therapy system, that a digital signature on the digitally signed certificate corresponds to an authorized instruction by either: using a unique, implant-specific key to confirm the digital signature, wherein the implant-specific key is known only to the implanted therapy system and to the remote server, or using a public key maintained in the implanted therapy system to confirm the digital signature corresponds to a private key held by only the remote server; and modifying the date and/or time setting of the implanted therapy system based on the received date and/or time setting once the digital signature is verified.

A method of updating software and/or firmware of an implanted therapy system may include: receiving, in the implanted therapy system, a software and/or firmware update for the implanted therapy system with a digitally signed certificate, wherein the digitally signed certificate was provided by a remote server; verifying, by the implanted therapy system, that a digital signature on the digitally signed certificate corresponds to an authorized instruction by either: using a unique, implant-specific key to confirm the digital signature, wherein the implant-specific key is known only to the implanted therapy system and to the remote server, or using a public key maintained in the implanted therapy system to confirm the digital signature corresponds to a private key held by only the remote server; and updating the software and/or firmware of the implanted therapy system based on the received software and/or firmware update once the digital signature is verified.

An implantable therapy system for applying electrical stimulation to a nerve may include: one or more electrodes; and one or more processors in the implantable therapy system, the processor comprising a memory storing computer-program instructions, that, when executed by the one or more processors, perform a computer-implemented method comprising: receiving, in the implanted therapy system, a communication from a user with a digitally signed certificate, wherein the digitally signed certificate was provided to the user by a remote server after verifying that the user is a clinician who has provided a recognized user name and password to the remote server; verifying, by the implanted therapy system, that a digital signature on the digitally signed certificate corresponds to an authorized user by either: using a unique, implant-specific key to confirm the digital signature, wherein the implant-specific key is known only to the implanted therapy system and to the remote server, or using a public key maintained in the implanted therapy system to confirm the digital signature corresponds to a private key held by only the remote server; and modifying operation of the implanted therapy system based on the communication received from the authorized user.

The methods and apparatuses (e.g., systems, devices, etc.) described herein may be used in whole or in part, with the methods and apparatuses described, e.g., in U.S. patent application Ser. No. 14/887,192, filed on Oct. 19, 2015 and titled, “NEURAL STIMULATION DEVICES AND SYSTEMS FOR TREATMENT OF CHRONIC INFLAMMATION”, Patent Cooperation Treaty (PCT) application no. PCT/US2016/032169, titled “EXTERNAL PROGRAMMER,” filed on May 12, 2016, and Patent Cooperation Treaty (PCT) application no. PCT/US2017/014453, titled “IMPLANTABLE MICROSTIMULATORS AND INDUCTIVE CHARGING SYSTEMS,” filed on Jan. 20, 2017. Each of these patent applications is herein incorporated by reference in its entirety.

### SUMMARY OF THE DISCLOSURE

The present invention relates generally to apparatuses (systems and devices) and methods for treating an inflammatory disease stimulation the NCAP. Any of the methods and apparatuses described herein may be configured for sub-diaphragmatic stimulation. Also described herein are methods of implanting a stimulation apparatus (including sub-diaphragmatic implantation of a stimulation apparatus) for NCAP stimulation, apparatuses for stimulation (including sub-diaphragmatic stimulation apparatuses for sub-diaphragmatic NCAP stimulation), and methods and apparatuses for noninvasively charging a stimulation apparatus, including but not limited to apparatuses and methods for sub-diaphragmatic NCAP stimulation.

Described herein are microstimulators (MSs, also referred to herein as microregulators or MRs) that may be implanted for stimulating a nerve, such as the vagus nerve. Any of these apparatuses may include a coil for receiving (and in some examples transmitting) information and/or for inductively charging the implanted device. The coil may be referred to as an antenna or inductive coil, or inductive coil antenna. In some examples the coil may be wrapped around a housing and/or a core which may be completely or partially ferromagnetic, which may modify (e.g., concentrate, direct) the magnetic field for effective charging and/or communication with a remote charger.

The apparatuses (devices and systems) and methods of using them described herein may incorporate some or all of the features of microstimulators (which may also be referred to as microcontrollers), nerve cuffs (“PODs”), chargers, and programmer/controllers described herein may be similar or identical to those described in U.S. patent application Ser. No. 12/874,171, titled “PRESCRIPTION PAD FOR TREATMENT OF INFLAMMATORY DISORDERS” and pending U.S. patent application Ser. No. 12/797,452, titled “NERVE CUFF WITH POCKET FOR LEADLESS STIMULATOR,” and U.S. patent application Ser. No. 14/887,192, filed Oct. 19, 2015.

The apparatuses (devices and systems) described herein may include chargers that are adapted to direct the magnetic field for communication with an implanted microstimulator. These chargers may include a high magnetic permeability material that shapes the magnetic field. In particular, the charger may be configured as a collar or wearable loop (belt, wristlet, anklet, necklace, etc.) that includes a magnetically permeable material to displace the density of the magnetic field axially “up” or “down” relative to the loop.

For example, a method of treating an inflammatory disease by sub-diaphragmatic stimulation of the vagus nerve may include: positioning a nerve cuff over a sub-diaphragmatic vagus nerve of a patient by longitudinally introducing the nerve cuff on the sub-diaphragmatic vagus nerve; positioning a microstimulator within the nerve cuff in electrical contact with the sub-diaphragmatic vagus nerve; sealing the microstimulator within the nerve cuff; applying electrical stimulation from the microstimulator to the sub-diaphragmatic vagus nerve to inhibit inflammation; and inductively charging the microstimulator from a belt worn around an abdominal region of the patient. The pillow-charging apparatuses described herein may be particularly well suited to charging implanted microstimulators that are implanted sub-diaphragmatically. Any of the pillow-charging apparatuses may be configured as mattress or mattress-covering devices that may be positioned at or below the patient's torso level when the patient is recumbent thereon.

The apparatuses (devices and systems) described herein may include cybersecurity measures to ensure 1) that clinicians/providers/prescribers can be authenticated and authorized to modify neurostimulation parameters (i.e., “digital prescriptions”), 2) that neurostimulation parameters (i.e., “digital prescriptions”) can be verified to be from an authentic source, 3) that the expiration of neurostimulation parameters (i.e., “digital prescriptions”) can be verified and enforced, and that tampering with the firmware of the therapy system can be prevented.

### BRIEF DESCRIPTION OF THE DRAWINGS

FIG. 1 is a perspective view depicting one example of a nerve cuff with stimulation device implanted proximate a nerve.

FIG. 1A is a top view depicting the implanted nerve cuff with stimulation device of FIG. 1.

FIG. 1B is a top view depicting the implanted nerve cuff with stimulation device.

FIG. 2A shows one example of a system for modulating chronic inflammation including a leadless microstimulator (shown connected to the vagus nerve) and an external charger/controller.

FIG. 2B shows another example of a system for modulating chronic inflammation, including a microstimulator, external charger (“Energizer”), and clinician-facing software (“Prescription Pad”).

FIG. 3 illustrates a sequence for authorizing a secure connection between a clinician such as a physician and a therapy system.

FIG. 4 illustrates a sequence for verifying the authenticity of a digital prescription or stimulation parameters for a therapy system.

FIG. 5 illustrates a sequence for authenticating and establishing an accurate time on a therapy system.

FIG. 6 shows a method for determining whether a time update sent to the therapy system is from a reliable source and was delivered in a timely manner.

FIG. 7 shows a backup mechanism for delivering electrical stimulation to a patient in scenarios where the internal clock of the implant is not accurate or known.

FIG. 8 illustrates a sequence for authenticating a firmware update of a therapy system as being from a trusted and reliable source.

FIGS. 9A-9G illustrate various display screens of a patient-facing application for a therapy system that keep the patient aware of expired or soon-to-be expired neurostimulation parameters (i.e., “digital prescriptions”).

FIG. 10 provides an example of an interface for a clinician-facing application where a physician or clinician could control and renew expiring digital prescriptions.

FIG. 11 is a flowchart that describes a prescription refill authorization and transmission sequence for a therapy system where the patient does not need to physically visit a clinic.

### DETAILED DESCRIPTION

Described herein are methods and apparatuses (e.g., devices and systems) for vagus nerve stimulation to treat inflammation. Any of the apparatuses and methods described herein may be used with any vagus nerve stimulation (e.g., with any micro-stimulator), but may be adapted in particular for use in sub-diaphragmatic vagus nerve stimulation. In particular, the methods and apparatuses may be used to stimulate the sub-diaphragmatic vagus nerve to treat inflammation and/or inflammatory disorders such as hay fever, atherosclerosis, arthritis (e.g., rheumatoid, bursitis, gouty arthritis, polymyalgia rheumatic, etc.), asthma, autoimmune diseases, chronic inflammation, chronic prostatitis, glomerulonephritis, nephritis, inflammatory bowel diseases, pelvic inflammatory disease, reperfusion injury, transplant rejection, vasculitis, myocarditis, colitis, etc. The apparatus may be a microstimulator (also referred to herein as a “MS”, “nerve stimulator”, “microcontroller”, “MC” or simply “implant”) that is configured to deliver appropriate electrical stimulation to a nerve. The electrical stimulation can modulate the activity of the nerve and cause a wide variety of effects. For example, electrical stimulation of the vagus nerve can result in a reduction of inflammation through activation of the cholinergic anti-inflammatory pathway. The microstimulator may be applied by itself or within or as part of a nerve cuff (referred to herein as a “protection and orientation device” or POD). The microstimulator (“MR”) or POD or combined MR/POD may be specifically adapted for use in the abdomen and placement sub-diaphragmatically. For example, the apparatus may be tethered or tetherable to prevent migration or “loss” of the apparatus within the abdomen. MS, POD or MS/POD may also be adapted for placement specifically on the sub-diaphragmatic region, including but not limited to the sub-diaphragmatic vagus Nerve.

When configured to sub-diaphragmatic vagus nerve stimulation, the apparatuses described herein may be preferred over carotid vagus nerve application. In such examples, a single implant (e.g., single leadless microstimulator, including those described herein) may be adapted for sub-diaphragmatic implantation. Such implants may be adapted for sub-diaphragmatic implantation by including one or more features including: a location and/or orientation emitter (configured to emit a wireless signal indicating implant location and orientation, particularly of an inductive coil(s) within the implant), multiple inductive coils for communication (including charging), a controller adapted to deliver a large simulation dose (e.g., a single pulse or bursts of pulses having between 6.5 and 20 V for a dose duration of between 0.1 second and 1000 seconds (e.g., between 0.3 s and 500 s, between 0.5 s and 100 s, etc.), followed by a low-power, “off” time during which the implant may not apply stimulation; this off time may be between 1 hour and 48 hours (e.g., between 2 hours and 48 hours, between 3 hours and 48 hours, between 4 hours and 36 hours, greater than 2 hours, greater than 3 hours, greater than 4 hours, etc.). The multiple inductive coils may be arranged as a biaxial or tri-axial array of coils. The coils may be arranged (e.g., wrapped, embedded, etc.) on an outer housing of the microstimulator. As will be described in greater detail below, any of these implants may include a magnetically permeable material. Any of these implants may also communicate with a charger which may be worn (e.g., around the abdomen, as a belt, sash, pant, bandolier, etc.) or configured for lying atop (e.g., pad, such as a mattress, pillow, etc.).

In general, the methods and apparatuses described herein for sub-diaphragmatic stimulation may be used with (or as part of) a laparoscopic surgical approach to placing MR/POD, e.g., on the posterior sub-diaphragmatic vagus nerve. In some examples the methods and apparatuses may be used as part of a Natural orifice transluminal endoscopic surgery (NOTES) procedure.

Examples of microstimulators and apparatuses for holding them onto the sub-diaphragmatic vagus nerve (e.g., a protection and orientation or “POD” device, also referred to herein as a nerve cuff) for use in sub-diaphragmatic stimulation of the NCAP are described herein as well. The methods and apparatuses described herein that are specific to sub-diaphragmatic NCAP stimulation have, in preliminary work, shown many advantages over traditional cervical Vagal placement. For example, these methods and apparatuses typically have fewer adverse events caused by stimulation. Further, these methods and devices may be less safety and time critical, therefore not requiring emergency shutoff as often or as precisely as cervical vagus stimulation. In addition, fewer cardiac effects have been seen with sub-diaphragmatic placement, and no laryngeal adverse events. Finally, there may be a substantial reduction in undesirable muscle stimulation and resulting pain.

The methods and apparatuses described herein may also alleviate the requirement for titration of stimulation patterns, due to a large predicted therapy window that may be used with sub-diaphragmatic stimulation of the NCAP pathway. This may also prevent or minimize postoperative pain. Sub-diaphragmatic stimulation may also reduce the risk of hemorrhaging due to insertion of the implant. Finally, the resulting microstimulator devices may be made larger, allowing greater energy storage and requiring less frequent charging.

In practice, the microstimulator for use in sub-diaphragmatic NCAP stimulation may be inserted onto a nerve forming a portion of the NCAP pathway below the diaphragm by any appropriate method. In particular, it may be helpful to connect (and tether) a microstimulator onto the posterior sub-diaphragmatic vagus nerve.

For example, a device such as the ones described below, or adapted from these devices, may be inserted by first creating several small incisions (0.5-1.5 cm) in abdomen and insufflating the abdomen with carbon dioxide gas. Two or more trocars may be inserted for access and/or to illuminate the surgical site. It may be helpful to displace internal organs such as the liver with a retractor to expose the posterior sub-diaphragmatic vagus nerve. Once exposed, the nerve may be separated from the tissue so that a POD may be placed under nerve, e.g., by longitudinally introducing a POD on nerve. Once the POD is applied, the microstimulator (MR) may be introduced into the POD, e.g., by separating the seam of the POD. The MR may then be sealed into the POD. The MR and POD may be any of those shown and described in Part II, below, or adapted specifically for sub-diaphragmatic implantation and operation. For example, the MR/POD may be configured to be tethered or attached within the abdominal cavity to prevent migration that may alter the position and/or orientation of the apparatus. This may be a particularly acute issue for sub-diaphragmatic implantation compared to cervical implantation. For example, a POD and/or MR may include one or more clips, anchors, and/or filaments for anchoring/tethering the device such as a polymer filament holding component, or the like.

In general, methods and apparatuses described herein may provide end-to-end secure neurostimulation parameters (i.e., “digital prescriptions”) that are tamper resistant, have the potential to expire, cannot be arbitrarily duplicated, and are ensured to be authentically issued only by the licensed issuer. The secure digital prescriptions described herein are digital scripts or instructions to be executed by a medical device, that includes a set of parameters enabling the authenticated delivery of “digital doses”. As used herein, “digital doses” can refer to a single dispensation of an electronic drug or therapy from a medical device with pre-determined parameters including duration, amplitude, pulse-width, frequency, maximum allotted doses, time of day or schedule details for when the digital doses are to be delivered to the patient by the medical device.

Any of the “digital prescription” information described herein may indicate a range of set values for stimulation parameters, including, but not limited to stimulation amplitude. A digital prescription may also indicate a range of stimulation amplitudes (e.g., between a minimum acceptable amplitude and a maximum permitted amplitude) that may be used. In some cases the patient may adjust the actual applied dose up/down within this range. In some variations the dose may include a maximum stimulation dose, allowing a patient to adjust the actual applied dose up to this maximum, or down to any lower level (including off). In other cases, a maximum and lower boundary of amplitude may be specified, and the patient would be unable to adjust the stimulation parameters below the minimum permitted amplitude or above the maximum permitted amplitude.

As further described herein, systems and methods can provide automated digital dose reminders to the patient. The digital dose reminder can be presented automatically on the patient's wearable, mobile phone, tablet, PC, or any other digital device. In some examples, the reminder can indicate to the patient if the dose will automatically be delivered without further action by the patient, or if the dose is manual and requires action by the patient or a caregiver to activate the digital dose delivery by the medical device.

In general, methods and apparatuses described herein may monitor compliance with prescribed digital doses and treatment plans and enforce dose limits. For example, a treatment system that includes an implantable medical device and at least one external digital device, such as a smartphone, PC, tablet, wearable, etc., can provide reminders to the patient regarding the number of digital doses received over a given time period (e.g., doses received in a day), provide reminders to the patient regarding how many doses are prescribed over a given time period, caution the patient on doses delivered too close together or too far apart, and provide reminders to the patient regarding lack of compliance towards dosing schedules.

In general, the systems and methods provided herein also enable Internet-based (i.e., “cloud-based”) connectivity between patients, physicians, and/or insurers to enable prescription validation, issuance, patient monitoring, and reporting. Artificial intelligence/machine learning can further be implemented to continuously improve therapy and use existing collected data on inputs and outcomes to drive improvements in outcomes. Reminders can be provided to a patient and/or physician to encourage communication between the patient/physician and to encourage physical office visits to review and/or renew prescriptions and therapy parameters. Additionally, implantable devices/sensors and/or wearable devices/sensors can be used to determine patient outcomes directly or as a correlate with a known outcome in order to continually monitor patient progress. This progress can be communicated to physicians or insurers in real time to ensure that providers are aware of patient compliance and improving or declining health.

### Neurostimulators and PODS

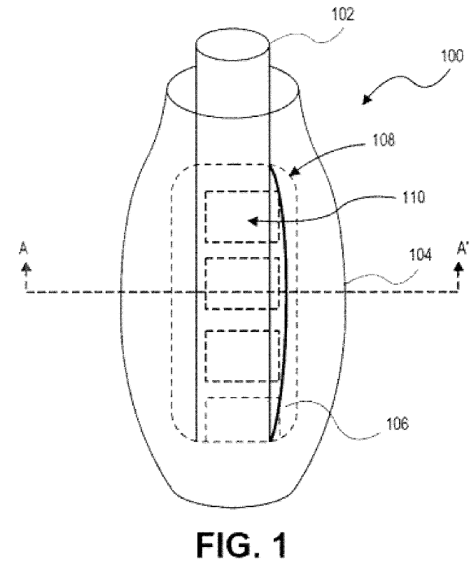

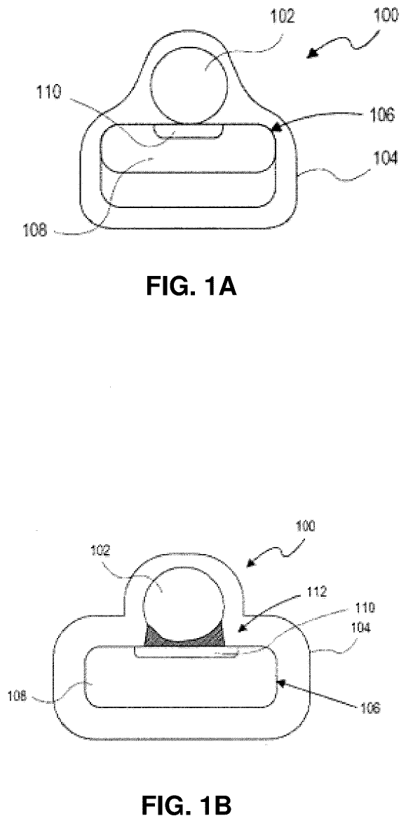

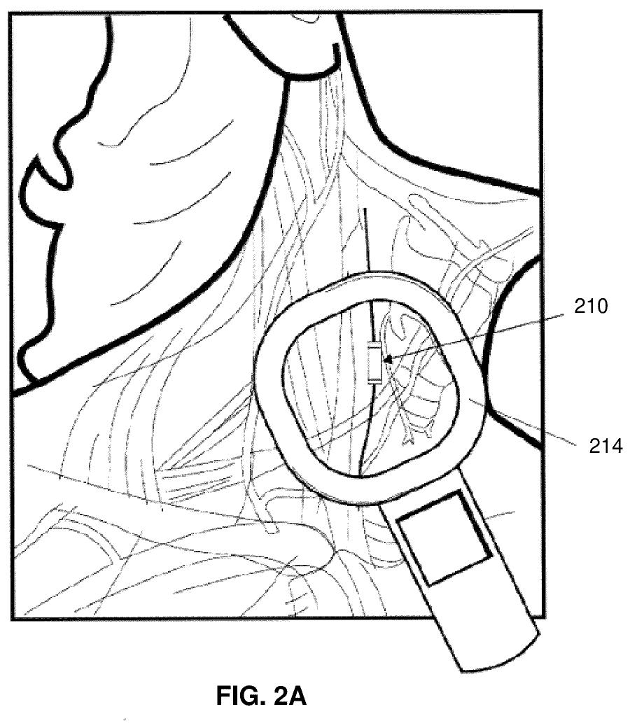

Referring to FIG. 1, one example of a nerve cuff 100 adapted for holding a stimulation device is coupled to a nerve 102. Although this cuff and microstimulator may be used for cervical vagus stimulation, any of the apparatuses described herein may be adapted for use in the sub-diaphragmatic region. In FIG. 1, the nerve 102 can comprise any nerve in the human body targeted for therapeutic treatment, such as, for example, the vagus nerve. Nerve cuff adapter 100 generally comprises an outer carrier or cuff 104 body that can comprise any of a variety of medical grade materials, such as, for example, Silastic™ brand silicone elastomers, or Tecothane™ polymer. Although this example is described for attaching the cervical region of a vagus nerve, it may be adapted as described herein for use in a sub-diaphragmatic site, including in particular the posterior sub-diaphragmatic vagus nerve. For example, the PODS described herein and/or the MR may include additional clips, tethers or the like for assisting in securing the apparatus to the posterior sub-diaphragmatic vagus nerve.

In general, a nerve cuff including a cuff 104 body having (or forming) a pouch or pocket 106 for removably receiving an active, implantable stimulation device 108 having one or more integrated, leadless electrodes 110 on a surface of stimulation device 108 proximate nerve 102. As illustrated in FIGS. 1 and 1A, nerve cuff 100 wraps around nerve 102 such that electrodes 110 are positioned proximate nerve 102.

Contacts or electrodes 110 can be positioned directly against nerve 102, as illustrated in FIG. 1A, or in close proximity to nerve 102, as illustrated in FIG. 1B. Referring specifically to FIG. 1B, close proximity of electrodes 110 and nerve 102 will leave a gap or space 112 that may naturally be filled with fluid or connective tissue. In one example of the invention, electrodes 110 and/or the inner surface of cuff body 104 can include optional steroid coatings to aid in reducing the local inflammatory response and high impedance tissue formation.

In one example, the pocket 106 for containing the stimulation device 108 is defined by the open space between the nerve 102 and the inner surface of the cuff body 104. Stimulation device 108 can be passively retained within pocket 106 by the cuff body 104, or can be actively retained on cuff body with fastening means, such as, for example, sutures. In other examples, pocket 106 can comprise a pouch-like structure attached to cuff body 104 into which stimulation device 108 can be inserted. Stimulation device 108 can be passively retained within a pouch-like pocket by simply inserting the device 108 into the pocket or can be actively retained with fastening means. A pouch-like pocket can be positioned either in the interior or on the exterior of cuff body 104. Pouch-like pocket 106 and/or cuff body 104 can include access openings to allow electrodes to be positioned directly proximate or adjacent to nerve 102.

Cuff body 104 can have a constant thickness or a varying thickness. The thickness of cuff body 104 can be determined to reduce the palpable profile of the device once the stimulation device is inserted. In one example, the thickness of cuff body can range from about 1 to about 30 mils, or from about 5 to about 20 mils. In one example, cuff 104 can have a greater thickness at a top and bottom portion of the cuff and a smaller thickness in a middle portion where the stimulation device is contained.

The nerve cuffs described herein may be opened and positioned around the nerve, for example, by splitting them open along a slit or hinge region. The device may be configured so that they have sufficient resiliency to close themselves, or remain closed if the edges of the slit region are brought together. Thus, the device may have a shape memory property that encourages them to close. In some examples, as already mentioned, it may be useful to hold them closed, at least temporarily, once they have been positioned over a nerve and the microstimulator has been positioned within the pocket. Thus, the device may include one or more closure elements. For example, the device may include a suture hole or passage for suturing the device closed. In some examples the nerve cuff includes a button or other fastener element. In some examples, the device may be sutured close with a dissolvable suture. A few weeks or months after insertion, the nerve cuff may be encapsulated or engulfed by the surrounding tissue, and will be held closed by this encapsulation. Thus, the dissolvable sutures merely keep the cuff closed for initial anchoring before biointegration and encapsulation occurs.

Any of the nerve cuffs described herein may also include one or more external leads or contacts facing the outside of the nerve cuff body, which may be used to stimulate tissues outside of the nerve cuff, and not just the nerve within the channel through the cuff. Such external leads may be used for sensing in addition to (or instead of) stimulation. For example, these electrical contacts may be used to sense other physiological events such as muscle stimulation and/or cardiac function. These signals can be applied to aid synchronization of target nerve stimulation to minimize artifacts of target stimulation. Such signals may be too faint for reliable remote sensing, however the position of the microstimulator (insulated within the housing of the nerve cuff) may allow accurate and reliable sensing.

In general, the nerve cuff body may be electrically insulating, preventing leakage of charge from the microstimulator during operation. In some examples the nerve cuff includes shielding or insulation sufficient to electrically insulate the microstimulator within the nerve cuff body. Shielding material may particularly include electrically insulative materials, including polymeric insulators.

Systems for electrically stimulating one or more nerves to treat chronic inflammation may include an implantable, wireless microstimulator such as those described herein and an external charging device (which may be referred to as a charging wand, charger, or energizer). In some examples the system also includes a controller such as a “prescription pad” that helps control and regulate the dose delivered by the system. The microstimulator may be secured in position using a securing device (which may be referred to as a “POD”) to hold the microstimulator in position around or adjacent to a nerve. These microstimulators are designed and adapted for treatment of chronic inflammation, and may be configured specifically for such use. Thus, an implantable microstimulator may be small, and adapted for the low duty-cycle stimulation to modulate inflammation. For example, the implantable microstimulator may hold a relatively small amount of power over weeks or even months and discharge it at a rate sufficient to modulate the anti-inflammatory pathway without significantly depressing heart rate or triggering any number of unwanted effects from the vagus nerve or other neural connections. Any of the nerves of the inflammatory reflex, including the vagus nerve, may be treated as described herein using the systems described.

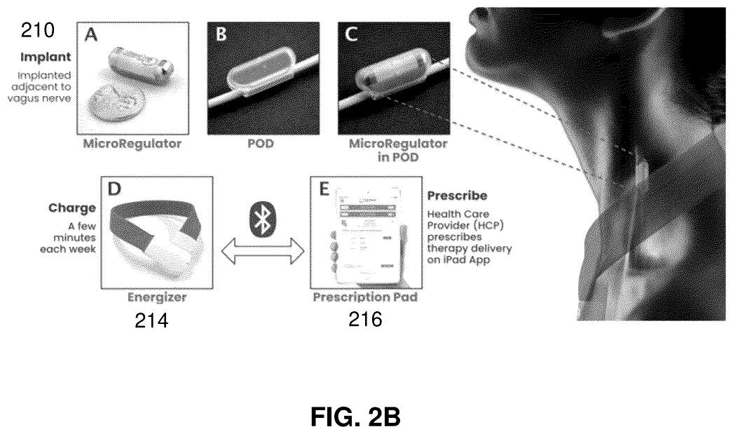

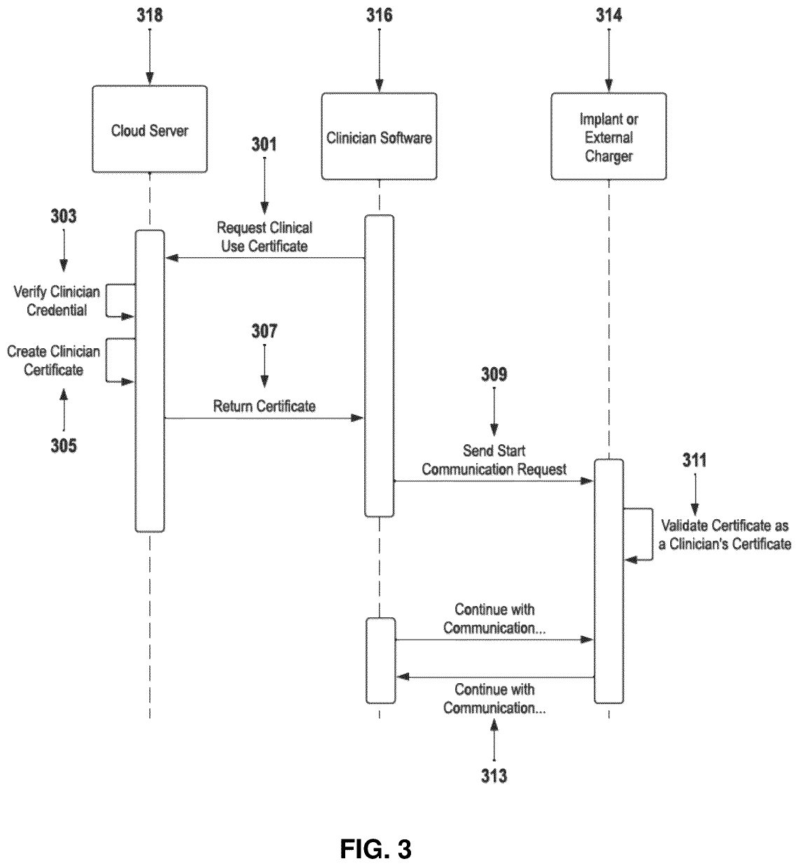

For example, FIG. 2A illustrates one example of a system for treating chronic inflammation that includes a microstimulator contained in POD that is mounted on cervical vagus nerve and charged a programmed by an external charger/programmer unit. This example of a system includes a microstimulator 210 that has been implanted to contact the vagus nerve as shown. The implant may be programmed, controlled and/or charged by a charger/controller 214 device. In this example the charger/controller is a loop with a wand region.

FIG. 2B shows another example of a system for treating chronic inflammation that also includes an implantable microstimulator or microregulator 210. The system can include A) the implant next to a penny for size reference, B) a POD for configured to hold the implant, and C) the implant inserted into a POD to hold it in position relative to a nerve. The system can further include a charging device (“energizer” 214) configured as a collar to be worn around the subject's neck and charge the implant. Optionally, the system may include a prescription pad 216 which may be a separate dedicated device or part of a mobile or other handheld device (e.g., an application to run on a handheld device) configured to communicate with the implant and/or the energizer. In some examples, the prescription pad can comprise a smartphone or tablet running, for example, an Android or iOS operating system. The systems described herein may also be referred to as systems for the neural stimulation of the cholinergic anti-inflammatory pathway (NCAP). These systems may be configured as chronic implantable systems. In some examples, the systems are configured to treat acutely (e.g., acute may include 8 hours or less), sub-acutely (expected to occur for fewer than 30 days), or chronically (expected to occur for more than 30 days).

In general, the systems described herein may be configured to apply electrical stimulation at a minimum level necessary to modulate the inflammatory reflex (e.g., modulating cytokine release) characterized by the Chronaxie and rheobase. Chronaxie typically refers to the minimum time over which an electric current double the strength of the rheobase needs to be applied in order to stimulate the neuron. Rheobase is the minimal electrical current of infinite duration that results in an action potential. As used herein, cytokines refer to a category of signaling proteins and glycoproteins that, like hormones and neurotransmitters, are used extensively in cellular communication.

The NCAP Systems described herein are typically intended for the treatment of chronic inflammation through the use of implanted neural stimulation devices (microstimulators) to affect the Neural Stimulation of the Cholinergic Anti-inflammatory Pathway (NCAP) as a potential therapeutic intervention for rheumatologic and other inflammation-mediated diseases and disorders. Neurostimulation of the Cholinergic Anti-inflammatory Pathway (NCAP) has been shown to modulate inflammation. Thus, the treatment and management of symptoms manifested from the onset of disease (e.g., inflammatory disease) is based upon the concept of modulating the Cholinergic Anti-inflammatory Pathway. The NCAP pathway normally maintains precise restraint of the circulating immune cells. As used herein, the CAP is a reflex that utilizes cholinergic nerve signals traveling via the vagus nerve between the brain, chemoreceptors, and the reticuloendothelial system (e.g., spleen, liver). Local release of pro-inflammatory cytokines (e.g., tumor necrosis factor or TNF) from resident immune cells is inhibited by the efferent, or indirectly by afferent vagus nerve signals. NCAP causes important changes in the function and microenvironment of the spleen, liver and other reticuloendothelial organs. Leukocytes which circulate systemically become “educated” as they traverse the liver and spleen are thereby functionally down regulated by the affected environment of the reticuloendothelial system. This effect can potentially occur even in the absence of an inflammatory condition.

Under this model, remote inflammation is then dampened by down-regulated cytokine levels. Stimulation of the vagus nerve with a specific regiment of electrical pulses regulates production of pro-inflammatory cytokines. In-turn, the down regulation of these cytokines may reduce localized inflammation in joints and other organs of patients with autoimmune and inflammatory disorders.

The NCAP System includes a neurostimulator that may trigger the CAP by stimulating the cervical vagus nerve. The NCAP System issues a timed burst of current controlled pulses with sufficient amplitude to trigger the CAP at a particular interval. These two parameters, Dose Amplitude and Dose Interval, may be used by a clinician to adjust the device. For example, the clinician may set the Dose Amplitude by modifying the current level. The Dose Interval may be set by changing the duration between Doses (e.g. 12, 24, 48 hours).

In some examples, dose amplitude may be set to within the Therapy Window. The Therapy window is defined as the lower limit of current necessary to trigger the CAP, and the upper limit is the level at which the Patient feels uncomfortable. The lower limit is called the Threshold (T), and the uncomfortable level is called Upper Comfort Level (UCL).

Dose Amplitude thresholds are nonlinearly dependent upon Current (I), Pulse width (PW), Pulse Frequency (PF), and Burst Duration (BD). Amplitude is primarily set by charge (Q), that is Current (I)×Pulse width (PW). In neurostimulation applications current has the most linear relationship when determining thresholds and working within the therapy window. Therefore, the clinician may modify Dose Amplitude by modifying current. The other parameters are held to experimentally determined defaults. Pulse width is selected to be narrow enough to minimize muscle recruitment and wide enough to be well above the chronaxie of the targeted neurons. Stimulus duration and pulse frequency was determined experimentally in Preclinical work.

Dose Interval may be specific for particular diseases and the intensity of diseases experienced by a patient. Our initial research has indicated that the cervical portion of the vagus nerve may be an ideal anatomic location for delivery of stimulation. The nerve runs through the carotid sheath parallel to the internal jugular vein and carotid artery. At this location, excitation thresholds for the vagus are low, and the nerve is surgically accessible. We have not found any significant difference in biomarker modulation (e.g., modulation of cytokines) between right and left. Even though the right vagus is thought to have lower thresholds than the left in triggering cardiac dysrhythmias, the thresholds necessary for NCAP are much lower than those expected to cause such dysrhythmias. Therefore a device delivering NCAP can safely be applied to either the right or left vagus.

Thus, any examples of the systems described herein (e.g., the NCAP system) may be implemented with a Cuff, Lead and Implantable Pulse Generation (IPG), or a Leadless Cuff. The preferred implementation is a leadless cuff implemented by a microstimulator with integral electrode contacts in intimate contact with the nerve and contained within a Protection and Orientation Device (POD). The POD may form a current shield, hold the microstimulator into place against the vagus nerve, and extend the microstimulator integral contacts with integral contacts in the POD itself. The POD is typically a polymer shell that encapsulates a microstimulator implant and that allows a nerve to run through the interior against the shell wall parallel to the length of the microstimulator implant. Within the shell of the POD, the microstimulator implant remains fixed against the vagus nerve so the electrodes remain in contact with the nerve. The POD anchors the implant in place and prevents the implant from rotating or separating from the nerve, as well as maintaining contact between the electrodes and the nerve and preserving the orientation as necessary for efficient external charging of the microstimulator battery.

As described in more detail in USSN Ser. No. 12/874,171 (titled “PRESCRIPTION PAD FOR TREATMENT OF INFLAMMATORY DISORDERS”), previously incorporated by reference in its entirety, the Prescription Pad may incorporate workflows in a simplified interface and provide data collection facilities that can be transferred to an external database utilizing commercially robust and compliant methods and procedures. In use, the system may be recommended for use by a clinician after assessing a patient; the clinician may determine that treatment of chronic inflammation is warranted. The clinician may then refer the patient to an interventional doctor to implant the microstimulator. Thereafter then clinician (or another clinician) may monitor the patient and adjust the device via a wireless programmer (e.g. Prescription Pad). The clinician may be trained in the diagnosis and treatment procedures for autoimmune and inflammatory disorders; the interventional placement of the system may be performed by a surgeon trained in the implantation of active neurostimulation devices, with a sufficient depth of knowledge and experience regarding cervical and vagal anatomy, experienced in performing surgical dissections in and around the carotid sheath.

The system may output signals, including diagnostics, historical treatment schedules, or the like. The clinician may adjust the device during flares and/or during routine visits. Examples of implantation of the microstimulator were provided in U.S. Ser. No. 12/874,171. For example, the implant may be inserted by making an incision in the skin (e.g., ˜3 cm) along Lange's crease between the Facial Vein and the Omohyoid muscle, reflecting the Sternocleidomastoid and gaining access to the carotid sheath. The IJV may be displaced, and the vagus may be dissected from the carotid wall (≤2 cm). A sizing tool may be used to measure the vagus, and an appropriate Microstimulator and POD Kit (small, medium, large) may be selected. The POD may then be inserted under nerve with the POD opening facing the surgeon, so that the microstimulator can be inserted inside POD so that the microstimulator contacts capture the vagus. The POD may then be sutured shut. In some examples a Surgical Tester may be used to activate the microstimulator and perform system integrity and impedance checks, and shut the microstimulator off, during or after the implantation. In other examples the surgical tester may be unnecessary, as described in greater detail below.

A physician or patient may use the Patient Charger to activate the microstimulator, perform integrity checks, and assure sufficient battery reserve exists. Electrodes may be conditioned with sub-threshold current and impedances may be measured. A Physician or patient may charge the microstimulator. In some examples a separate charger (e.g., an “energizer”) may be used by the patient directly, separate from the controller the physician may use. Alternatively, the patient controller may include controls for operation by a physician; the system may lock out non-physicians (e.g., those not having a key, code, or other security pass) from operating or modifying the controls.

In general, a physician may establish safe dosage levels. The physician may slowly increment current level to establish a maximum limit (Upper Comfort Limit). This current level may be used to set the Dosage Level. The exact procedure may be determined during this clinical phase.

The Physician may also specify dosing parameters that specify dosage levels and dosage intervals. The device may contain several concurrent dosing programs which may be used to acclimate the patient to stimulus, gradually increase dosage until efficacy is achieved, reset tachyphylaxis, or deal with unique patient situations.

### Charger

In some examples, the magnetic properties of the charger, and in particular, the housing surrounding the charger, may be modified to shape the field emitted by the charger for inductively charging the implant. In general, charging of the implant (e.g., inductively charging) may depend in part on the orientation and position of the implant relative to the charging field. The methods and apparatuses described herein may improve the relationship between the inductive field and the implant. For example, the charger, which may be a wearable charger such a collar, necklace, or the like, or it may be hand-held charger, may be configured (and/or the charging portion of the implant may be configured) to raise the inductance and thus the quality factor (Q), potentially providing a greater power transfer between the applied charging field and the implant.

For example, the charger housing may include a material having a high magnetic field permeability, such as a ferromagnetic ceramic material (ferrite), ferrite-filled polymer, ferrite-embedded polymer, alloys of iron and nickel (e.g., commercially sold as MUMETAL and PERMALLOY) and the like. These high-magnetic-permeability materials may concentrate and direct the magnetic field up from the charger (when worn on the neck, for example) towards the implant, and may help target the field in the neck and for reception by the implanted microcontroller.

For example, the microcontroller may be implanted so that it is generally oriented up and to the right, as shown in FIG. 2B. Thus, a charger (configured as a wearable collar) may be configured by the use of a high-magnetic field permeability material to direct the field up from the charger (e.g., collar) toward the implant. The higher (denser) field may also be shifted the right (patient's right) as well.

By steering the field, e.g., up from the collar, the collar may be more comfortably worn around the base of the person's neck. This reduces the importance of placing the charger closer (e.g., directly over and/or adjacent to) the implant, which may be located up to several inches above the base of the neck, as show on in FIG. 2B. Further, the orientation of the field may be adapted using the high magnetic permeability material so that it is oriented appropriately to charge the implant.

Examples of high magnetic permeability materials include ferrites, ferrite polymer composites, ferrite filled polymers, ferrite loaded rubber, and a ferrite tape. Other high magnetic permeability materials that may be used include Mu-metal, iron, steel, and various metal alloys. In some examples, the high magnetic permeability materials have a relatively magnetic permeability that is greater than 10, 100, 1000, 10,000 or 100,000 times the magnetic permeability of a vacuum.

In some examples as described above, the charger can include a transmitting coil that is worn around the neck in order to charge a neurostimulator that is positioned on the cervical portion of the vagus nerve. If the patient is sitting or standing during the charging process, the charger may end up resting on the lower portion of the neck while the neurostimulator is implanted in a higher portion of the neck. The transmitting coil in the charger can generate an electromagnetic field with field lines that run through the loop. A high magnetic permeability material can be added around the transmitting coil to concentrate and direct the field lines towards the neurostimulator. For example, the high magnetic permeability material can be added to the upper inner surface of covering around the transmitting coil in order to concentrate and direct the field lines upwards and inwards towards a neurostimulator located in the neck and above transmitting coil. In other examples, the high magnetic permeability material can be added just to the inner surface of the covering that faces the skin, or the high magnetic permeability material can be incorporated into the entire covering.

Systems and methods are described herein that enable seamless communication and coordination between an implantable neurostimulator, external charging device, and clinician-facing software described above. This functionality enables control of the implantable neurostimulator with an external charging device or clinician software, and provides a deep insight into the status of the implantable neurostimulator, including views of the battery life, device diagnostics, and more. Furthermore, the system encourages compliance and on-time dosing through automated mechanisms and reminders.

The digital therapy system described herein can rely on a “digital prescriptions” to provide “digital doses” or therapy to the patient. In this system, stimulation parameters such as stimulation amplitude and duration from the implantable device can be treated like medication dosages. This digital prescription is determined by the patient's physician, and can include a limited number of doses, a schedule of when doses are to be delivered as well as the electrical parameters of the dose (e.g. the amplitude and duration of each dose). The digital prescription can be configured to expire, either upon completion of all prescribed doses or after a predetermined time (e.g., a prescription may be good for 1 month, 6 months, etc.). The number of doses delivered for a particular digital prescription can be tracked by the system (e.g., by maintaining a count of delivered doses in the implant, energizer, or prescription pad). In some implementations, the system can provide the option for prescription refills without the need to re-visit a physician's office. An interaction between a patient-facing, the Internet (i.e., “cloud”), and a clinician-facing application includes a mechanism for managing and renewing expiring or expired digital prescriptions to encourage continuous physician engagement and care reimbursement. The topology described herein can include the use of asymmetric or symmetric cryptography to guarantee refills come from an authentic and authorized source (e.g., an authorized physician using systems within the provider's network).

Physical consumable medications have a few advantages that digitally provided therapies, including electrical stimulation from implantable devices, could benefit from. Namely, those physical medications will eventually be consumed by the patient at which point the patient either needs to return to their pharmacy and/or doctor to obtain more medication. In other words, the patient must explicitly request a refill from their prescriber. These explicit requests for refills are extremely useful for physicians as it allows the physician to reevaluate the patient's care to determine if the medication and/or dosage is still appropriate for the patient. The physician can evaluate if other medications or therapies are needed, or if the patient needs additional diagnostic tests (e.g., blood tests) to ensure the prescribed medication is not causing side effects. Likewise, with the current reimbursement systems in the United States, these continued office visits are often critical for doctors to ensure they are being properly reimbursed for the care they provide.

Unfortunately, once an implant is activated, the patient often ceases to return to their physician for any sort of routine follow-up visits. Instead, the patients typically only show up at the clinic if something goes awry (and sometimes not even then). This is detrimental for both the patient and the physician. The patient is not getting the routine care they require and effectively forestalling treatment for problems that are much easier to deal with if caught early. The clinicians are not getting the opportunity to give their patients the best possible treatment, nor are they receiving reimbursement.

This disclosure provides systems and methods in which the digital or electrical “prescriptions” offered by the system are treated more like conventional medication in that they will have the capability of expiring. This is actually a relatively complicated technical challenge, as ensuring that “doses” of the neurostimulation can be neither counterfeited nor maliciously applied requires developing an authenticated and tamperproof system. This, in turn, requires application of modern and upgradable cryptographic techniques.

Any client software running on PC, tablet, phone, etc. is essentially inherently prone to reverse engineering, cloning, and tampering. This means that software accessed by the user cannot be the end-all of cybersecurity tasks (though it does include many mitigations to make tampering with the system significantly harder). Instead, critical authentication activities should be done on remote hardware—ideally the implant itself. However, in the case of the implant described above, the implant's considerably limited resources and lack of an internet connection can prevent everything from being processed on the implant. Instead, according to the present disclosure, each cybersecurity task can be carefully considered and placed at the lowest level possible as resources permit. In the system described herein, some cybersecurity mitigations can occur on the implant, some can occur on the energizer or charger, some can occur in the user or patient-facing software (e.g., the prescription Pad), and some cybersecurity mitigations can occur in the cloud.

### Authorizing and Authenticating Clinicians

The systems and methods disclosed herein implement hardware and software safeguards to ensure that any users working with the system are properly authorized to do so. In the context of implantable neurostimulators, these systems and methods described herein may ensure that only properly licensed and vetted physicians or clinicians are able to create and prescribe dosing parameters for the implantable stimulator.

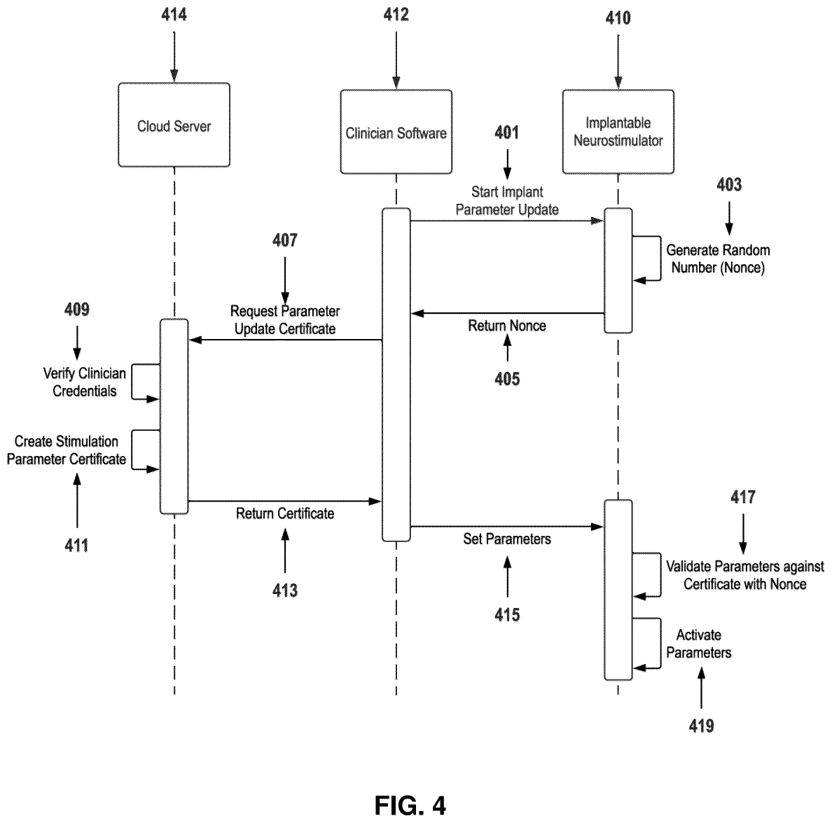

FIG. 3 illustrates a sequence for authorizing a secure connection between a provider such as a physician and a therapy system as discussed herein, including a therapy system including an implant or external charger 314, clinician-facing software 316, and a cloud (e.g., remote) server 318.

At step 301 of the sequence, a clinician or physician can use software 316 to request authorization to modify or send instructions/prescriptions to a patient's implantable device and therapy system. The request for authorization can be handled by the cloud server 316 which may require, for example, the clinician to login to an application or website with a username/password. In some implementations, the credentials of the clinician may be verified during creation of a new user account. In other implementations, clinicians are provided with verified login credentials when they undertake providing care to a patient with an implantable therapy system.

At step 303, the cloud server can verify authorization (e.g., verify the clinician's login credentials), and then at step 305, the cloud server can create a digital certificate with data that indicates it is valid for clinician use. Creation of this digital certificate is only possible with possession of one or more secret keys. The secret key(s) may, alternatively, be stored on more private and guarded servers, or certified hardware security modules.

At step 307, digital certificate(s) can then be returned to the clinician's software running. In some implementations, the digital certificate(s) may also authorize the use of the clinician's software itself. This may be implemented, for example, by “unlocking” certain clinician functions in the software such as progressing beyond the login screen, assigning new digital prescriptions, or updating/modifying existing prescriptions.

At step 309, the clinician or clinician software can attempt to begin communication with the implantable neurostimulator or its external charging system with a now-elevated clinical role. This role can include, for example, requests to transmit a new prescription, modify or access an existing prescription, access diagnostics or therapy information from the therapy system, etc.

At step 311, the implantable neurostimulator or external charging system, which comes preprogrammed with a corresponding public key to the private key from the private server (asymmetric encryption) or a shared secret key (symmetric encryption), can validate the certificate transmitted from the cloud, through the clinician's software, to the implant device or external charging device. If the user/clinician does not have the proper credentials, the devices can be configured to block all commands from external software that only a clinician should have access to (e.g., modifying stimulation parameters or accessing certain diagnostic data).

At step 313, the devices and the clinician's software can communicate bi-directionally to incorporate any new instructions from the clinician software into the therapy system. This communication can be wireless communication using proprietary communication schemes or standard technologies such as NFC, Bluetooth, Wi-Fi, cellular, etc.

### Verifying Authenticity of Neurostimulator Parameters

The therapy system disclosed herein employs safeguards to verify and ensure the authenticity of parameters to be delivered by the implant, including stimulation parameters (e.g., “digital prescriptions”) or other parameters such as battery charging, diagnostic collection modes, etc. This serves as a vital safeguard to prevent malicious stimulation or malicious parameters that may damage the device from being sent to a patient's implant, and also prevents patients from bypassing their doctor's prescribed dosing parameters. Additionally, the implant of the therapy system described may be a small size and may have limited processing power, which requires both a power and CPU efficient method for authenticating stimulation parameters.

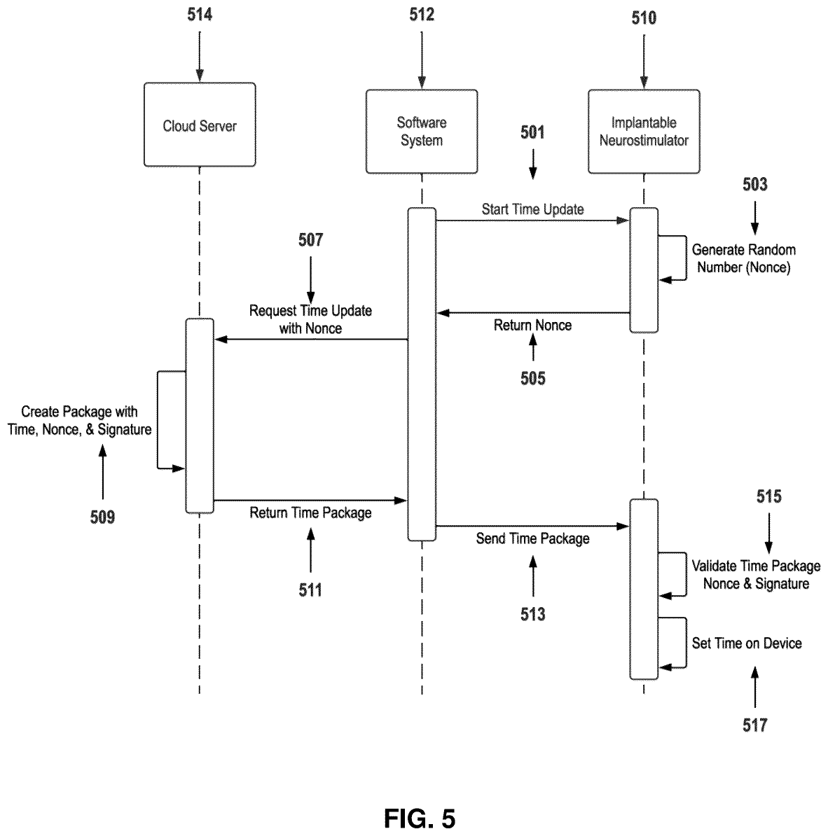

FIG. 4 illustrates a sequence for verifying the authenticity of implant parameters (including stimulation parameters) for a therapy system as discussed herein, including a therapy system including an implant 410, clinician software 412, and a cloud server 414.

At step 401 of the sequence, a clinician or physician can use software 412 to request an update to a patient's implantable device and therapy system. The update can comprise, for example, adding a new digital prescription to the therapy system, updating an existing digital prescription, adding or modifying stimulation parameters to be delivered by the therapy system, updates to battery charging routines, or any variety of other sensitive parameter updates.

At steps 403-405, the implant of the therapy system can receive the request for a parameter update. In return, the implant can be configured to generate, store, and return a random “nonce” number (e.g., a very large random number that will only be used once by the system). Although examples described herein discuss this communication as being between the clinician software and the implant, in other examples it should be understood that the clinician software can communicate with other components of the therapy system as intermediary devices, including an external charger.

At step 407, the clinician software 412 can request a signed implant device configuration parameter update certificate from the cloud server 414. In some implementations, this request may require, for example, the clinician to login to an application or website with a username/password or other credentials as described above. The request for the parameter update certificate will include, amongst its digitally signed data, the “nonce” to ensure the parameter update cannot simply be repeated at a later point in time.

At step 409, the cloud server can verify authorization (e.g., verify the clinician's login credentials), and, at step 411, generate a digitally signed parameter update certificate. Creation of this digital certificate is only possible with possession of one or more secret keys. The secret key(s) may, alternatively, be stored on more private and guarded servers, or certified hardware security modules.

At step 413, the cloud server can return a parameter update certificate, that is digitally signed, with a secret key, to the clinician software. This parameter certificate is then forwarded to the implantable neurostimulator in step 415.

At step 417, the implantable neurostimulator, which comes preprogrammed with a corresponding public key to the private key from the private server (asymmetric encryption) or a shared secret key (symmetric encryption), can validate the certificate's signature. The digital certificate, in addition to having a valid signature, must also include the “nonce” in its signed data. This ensures two things: 1) that the parameters were signed by the implant manufacturer, and 2) that the parameters have not expired nor originated from a previously-sent parameter update. After ensuring the parameters are valid and authentic, they are applied as active parameters on the implant per step 419.

### Ensuring Time Update Authenticity

The neurostimulation parameters or “digital prescriptions” described herein can include expiration dates at which point the therapy system will cease to apply stimulation to the patient based on the prescriber's instruction. These digital prescriptions with expiration dates can generally only work if the system checking for expiration has a reliable and secure source of time. The devices and methods described herein can implement safeguards to ensure a secure and reliable source of time to ensure that expired digital prescriptions cannot be used by the therapy system to deliver stimulation to a patient. Additionally, the methods and techniques discussed herein can also safeguard against situations in which the system itself cannot accurately determine time (e.g., in the case of a discharged battery in the implant).

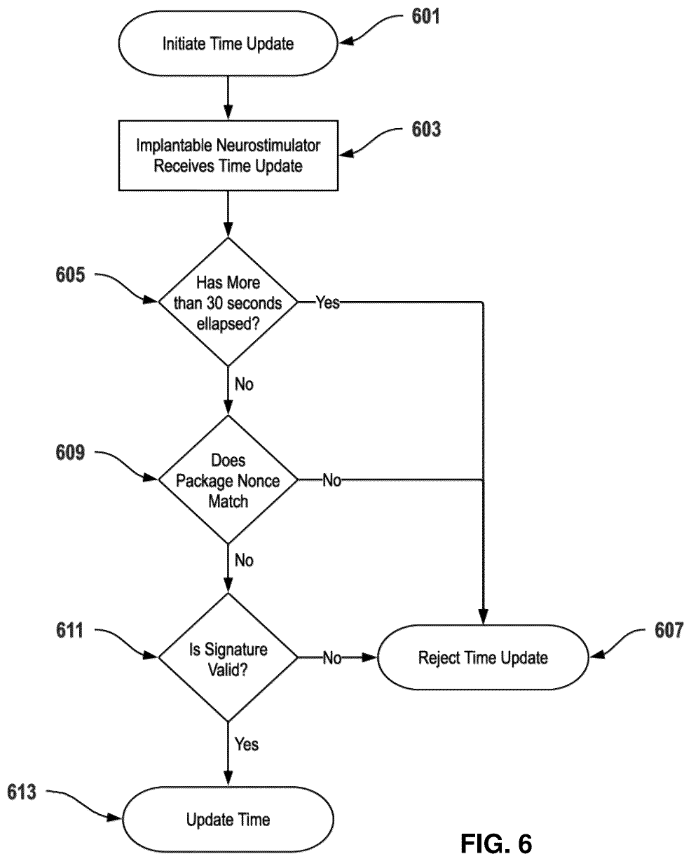

FIG. 5 illustrates a sequence for verifying an accurate time and expiration for a therapy system as discussed herein, including a therapy system including an implant 510, a software system (either patient-facing, or clinician-facing) 512, and a cloud server 514.

At step 501 of the sequence, the software system 512 can sync with a patient's implantable device and therapy system to start a time update. The time update can comprise, for example, confirming the accurate time in a therapy system to determine if a digital prescription is still valid or if it is expired. In some implementations, this time update is completed every time the software system is synced to the therapy system.

At steps 503-505, the implant or microregulator of the therapy system can receive the request for a time update. In return, the implant can be configured to generate, store, and return a random “nonce” number (e.g., a very large random number that will only be used once by the system). Although examples described herein discuss this communication as being between the software system and the implant, in other examples it should be understood that the software system can communicate with other components of the therapy system, including an external charger.

At step 507, the software system 512 can request a time package from the cloud server 514. In some implementations, this request may require, for example, the user to login to an application or website with a username/password or other credentials as described above. In other implementations, the request to the cloud server does not require login credentials and can occur automatically when the software system syncs with the implant.

At step 509, the cloud server will generate a digitally signed time update certificate. Creation of this digital certificate is only possible with possession of one or more secret keys. The secret key(s) may, alternatively, be stored on more private and guarded servers, or certified hardware security modules. The time that is returned in the digital certificate package will contain the current date and time according to the cloud server.

At step 511, the cloud server can return a time update certificate, that is digitally signed with a secret key, to the software system. This time update certificate is then forwarded to the implantable neurostimulator in step 513. Although examples described herein discuss this communication as being between the clinician software and the implant, in other examples it should be understood that the clinician software can communicate with other components of the therapy system as intermediary devices, including an external charger.

At step 515, the implantable neurostimulator, which comes preprogrammed with a corresponding public key to the private key from the private server (asymmetric encryption) or a shared secret key (symmetric encryption), can validate the time update certificate's signature. The digital certificate, in addition to having a valid signature, must also include the “nonce” in its signed data. This ensures two things: 1) that the time update was signed by the implant manufacturer, and 2) that the time update has not expired nor originated from a previously-sent time update. After ensuring the time update is valid and authentic, it is applied as the active time on the implant per step 517.

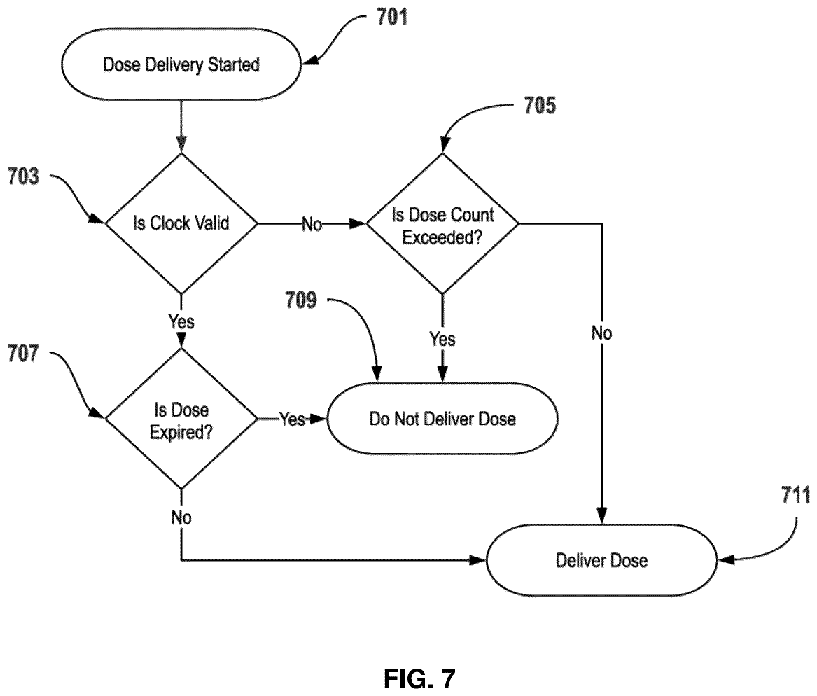

While the method described above with respect to FIG. 5 guarantees that the time package request comes from a reliable source, extra care must be taken to ensure that the time package was timely delivered and not intentionally or accidentally deferred or delayed. Referring to FIG. 6, a method is described for authenticating that a time package from a reliable source is timely delivered.

At operation 601 of FIG. 6, a time update can be initiated. At an operation 603, the time update can be received by the implant. This operation can correspond to operation 513 of the sequence diagram of FIG. 5.

The remaining operations 605-613 correspond to the validation operation 515 from the sequence diagram of FIG. 5. At operation 605 of FIG. 6, the therapy system determines the time period that has elapsed between the initiation of the time update and the return/receive of the time update (e.g., the time elapsed between steps 601 and 603). If this time period exceeds a pre-determined threshold (e.g., 5 seconds, 10 seconds, 15 seconds, etc.), then at step 607 the therapy system can reject the time update.

If the pre-determined threshold has not been exceeded at step 605, then at step 609 the therapy system can determine if the “nonce” number from the implant matches a corresponding nonce key in the time update package (e.g., the random number generated by the implant, and subsequently signed by the cloud server, in FIG. 5). If the nonce number does not match, then at step 607 the therapy system can reject the time update.

If the nonce number matches, then at step 611 the therapy system can determine if the digital signature in the time update is valid. If the pre-determined threshold is not exceeded at step 605, if the nonce matches at step 609, and if the signature is valid at step 611, then at operation 613 the time can be updated on the implant of the therapy system.

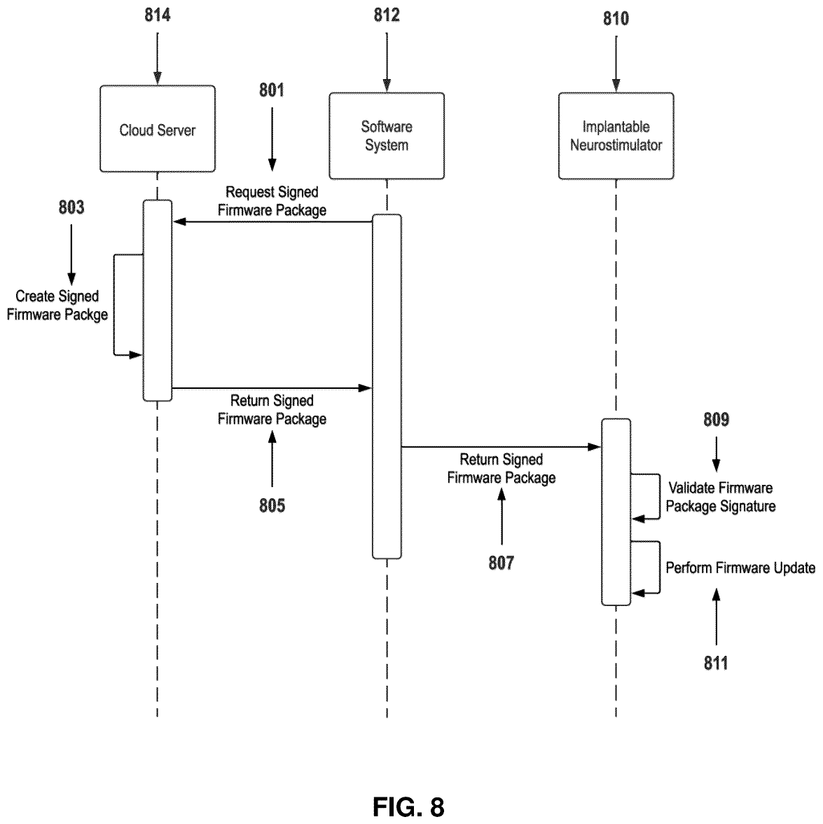

As described above, an issue can arise if the battery of the implant is allowed to fully drain or discharge, at which point the time/date within the implant will be inaccurate when charge is restored to the implant. In this scenario, if there is a request for delivery of stimulation without performing a time update, the implant must have a fallback method for determining whether or not to deliver the requested stimulation to the patient. Referring to FIG. 7, a flowchart is shown that provides a backup mechanism for delivering electrical stimulation to a patient in scenarios where the internal clock of the implant is not accurate or known.

At operation 701, the implant of the therapy system can initiate a dose delivery to a patient. At operation 703, the implant can determine if the clock is valid. The implant can determine if the clock is valid through multiple mechanisms, including invalid shutdown flags, seeing if the time is below a certain date, etc. If the implant determines that the clock or time update is not valid, then at step 705 the implant determines if a dose count for the current digital prescription is exceeded. As described above, the doses delivered can be tracked by the therapy system and a count is stored in nonvolatile memory, so even in the event that the clock is not updated, the number of delivered doses is still known. If the dose count is not exceeded, then at step 711 the dose can still be delivered by the implant of the therapy system. If, however, the dose count is exceeded, then at step 709 the implant will refuse to deliver the dose to the patient.

At step 707, if the clock is valid, then the system will refuse to deliver the dose if the prescription is expired or will deliver the dose if the prescription is not expired.

### Preventing Firmware Tampering

All of the safeguards mentioned above could be rendered moot if they could be bypassed by arbitrarily loading an entirely new set of firmware onto components of the therapy system. The unauthorized software, hardware and/or firmware could simply choose not to do any of the validation tasks described above. To mitigate against someone placing firmware on the device that bypasses other security controls, authentication schemes can be employed before the system allows a firmware update.

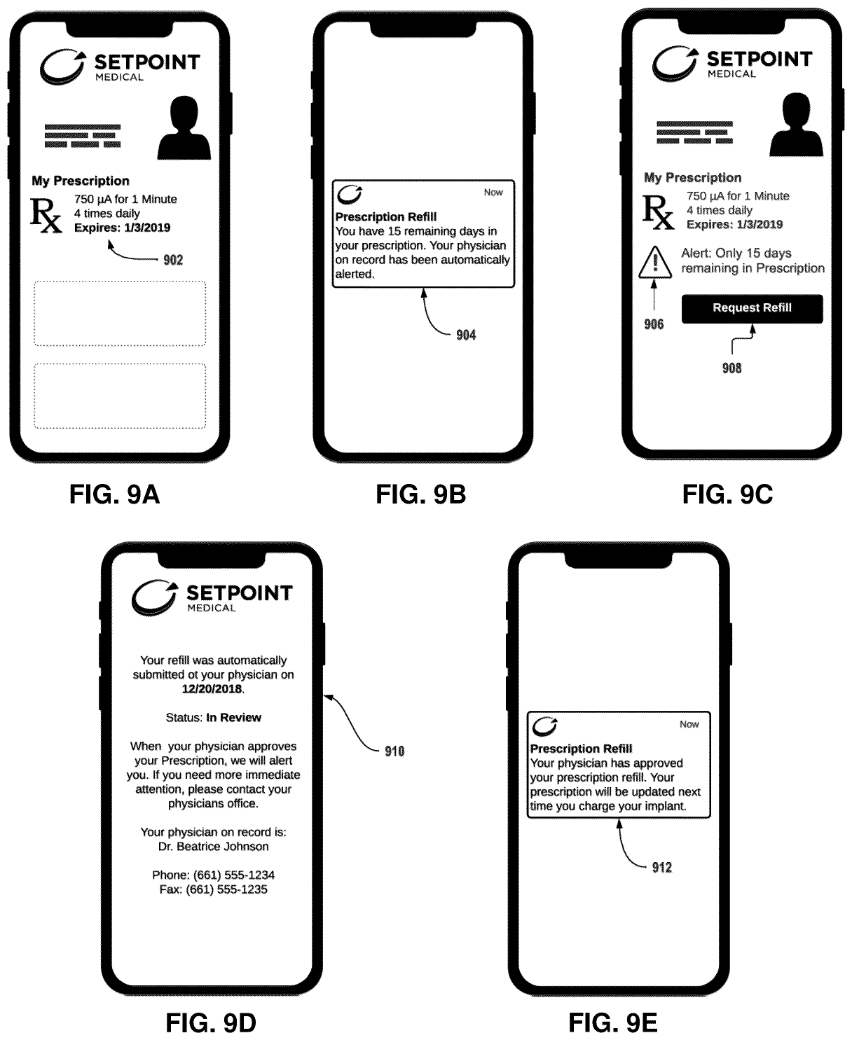

FIG. 8 illustrates a sequence for authorizing a firmware update of a therapy system as discussed herein, including a therapy system including an implant 810, a patient-facing or clinician-facing software system 812, and a cloud server 814.

At step 801 of the sequence, a user can use the software system 812 to request a signed firmware package to update the firmware of the therapy system. The request for firmware can be handled by the cloud server (remote server) 814 which may require, for example, the user to login to a website or application with a username/password. In some implementations, the credentials of the user may be verified during creation of a new user account. In other implementations, user is provided with verified login credentials when they undertake providing care to a patient with an implantable therapy system.

At step 803, the cloud server will create a digitally signed firmware package for a specific implantable neurostimulator. Creation of this digitally signed firmware package is only possible with possession of one or more secret keys. The secret key(s) may, alternatively, be stored on more private and guarded servers, or certified hardware security modules.

At step 805, the cloud server returns the signed firmware package to the software system 812. At step 807, the software system can transfer the firmware package to the implant 810. Although examples described herein discuss this communication as being between the clinician software and the implant, in other examples it should be understood that the clinician software can communicate with other components of the therapy system as intermediary devices, including an external charger.

At step 809, the implantable neurostimulator, which comes preprogrammed with a corresponding public key to the private key from the private server (asymmetric encryption) or a shared secret key (symmetric encryption), can validate the firmware update's digital signature. After ensuring the firmware update is valid and authentic, the new firmware can be activated on the implant, e.g., performing the firmware (or software) update 811.

### Secure Prescription Refills

It is often unnecessary for a patient to return to the clinic just to refill their physical prescriptions. Instead, the prescriber can simply call into the pharmacy (or use an integrated computer system) to order a refill a prescription for the patient. A similar system is implemented in the therapy system described herein. The therapy system can leverage a patient-facing smartphone application to refill these digital prescriptions. This section will detail an example form of how a patient-facing application and the prescriber-facing Prescription Pad software can work together to manage a patient's refills.

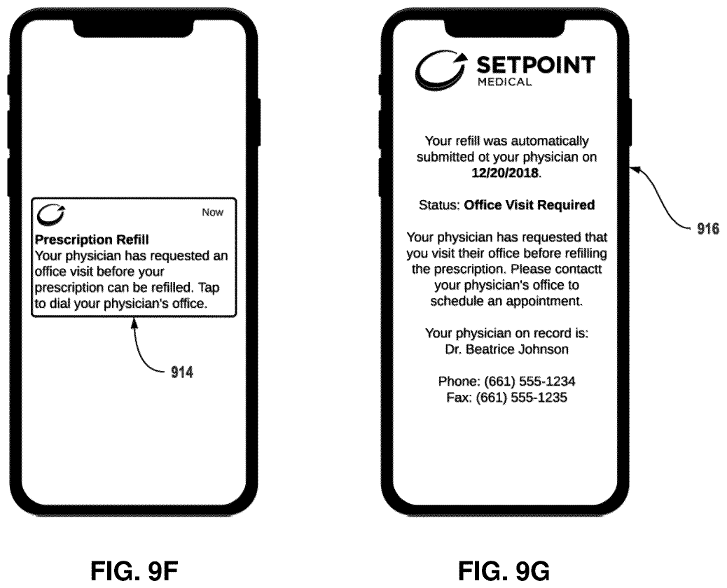

Referring to FIG. 9A, a dashboard screen of a patient-facing application of the digital therapy system can include prescription information 902 detailing the parameters and delivery schedule of therapy doses. In FIGS. 9B-9C, the patient-facing application can provide and an expiration notification interface 904 that can indicate to a user that a digital prescription has expired or will expire soon. The interface 904 in FIG. 9B can be shown on, for example, a lock screen or notification screen of a smart device, and the interface 906 in FIG. 9C can be shown on the dashboard screen of the patient-facing application. In the illustrated example, the expiration interface notifies the user that the prescription will expire in 15 days. The expiration interface provides the user with the option to submit a digital request to “refill” the digital prescription (i.e., request that more digital doses be allowed on the implantable neurostimulator). The prescription request can then be transmitted securely to the cloud, then to the physician/caregiver, using the topology described above.

If the patient requests a digital refill of the digital prescription, the system can send a notification to the prescriber alerting them that the patient has requested a refill. However, this sort of notification may be largely unnecessary in the therapy systems described herein as the system may know when a patient's prescription is nearing expiration before the patient ever requests a refill. In such a system, a “request refill” button 908 could act more as a non-functional “placebo” button and the prescribers would be notified of the need to refill without the patient ever having to ask for a refill.

At FIGS. 9D, the patient-facing application or the lock/notification screen of a smart device can provide an indication 910 to the user that the prescription refill request is being reviewed by the patient's physician. At FIGS. 9E, the lock/notification screen of a smart device or the patient-facing application can provide an indication 912 to the user whether the prescription has been refilled, or if further action is required, such as a visit to the physician's office for evaluation before refilling the prescription 914/916.

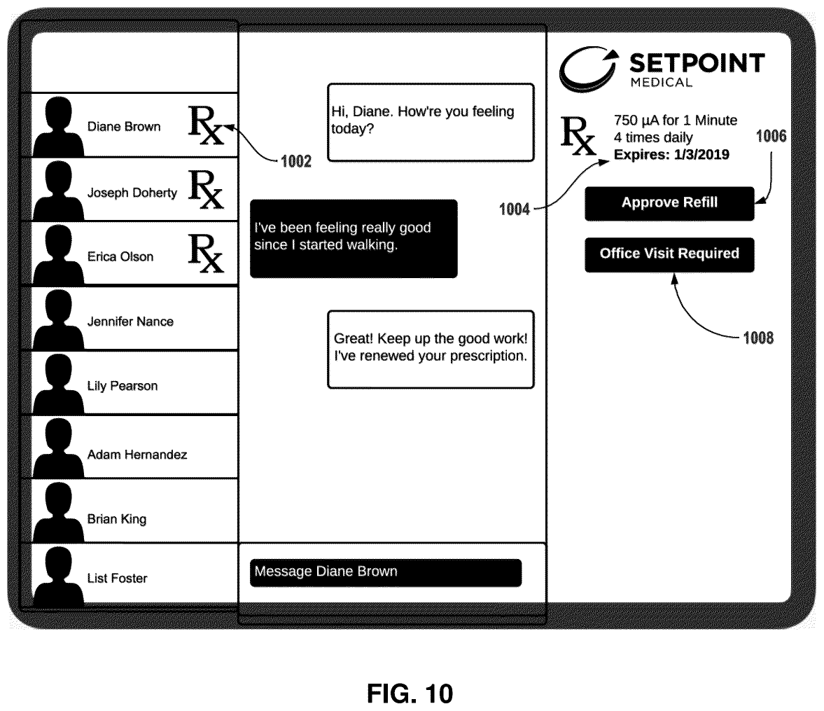

FIG. 10 provides an example of a clinician-facing software user interface. As shown, prescribers can see a list of their patients 1002 with the status of their prescriptions. In some implementations, patients with soon-to-expire prescriptions can be highlighted. Once a patient is selected, prescribers will have a quick access to seeing prescription details 1004 and options 1006 to refill a prescription or to deny the request 1008 (opting for an office visit instead).

The prescription refill sequence can implement similar techniques to the other validation methods as described herein. Once a prescription is approved by the prescriber, a certificate can be issued to the patient's phone or other devices, via the Internet, with the details of the prescription refill. This certificate can leverage the cryptographic concepts above to ensure the prescription refill is only valid for a particular patient's implant. At the first opportunity, the patient's device(s) will forward this prescription refill certificate to the implant.

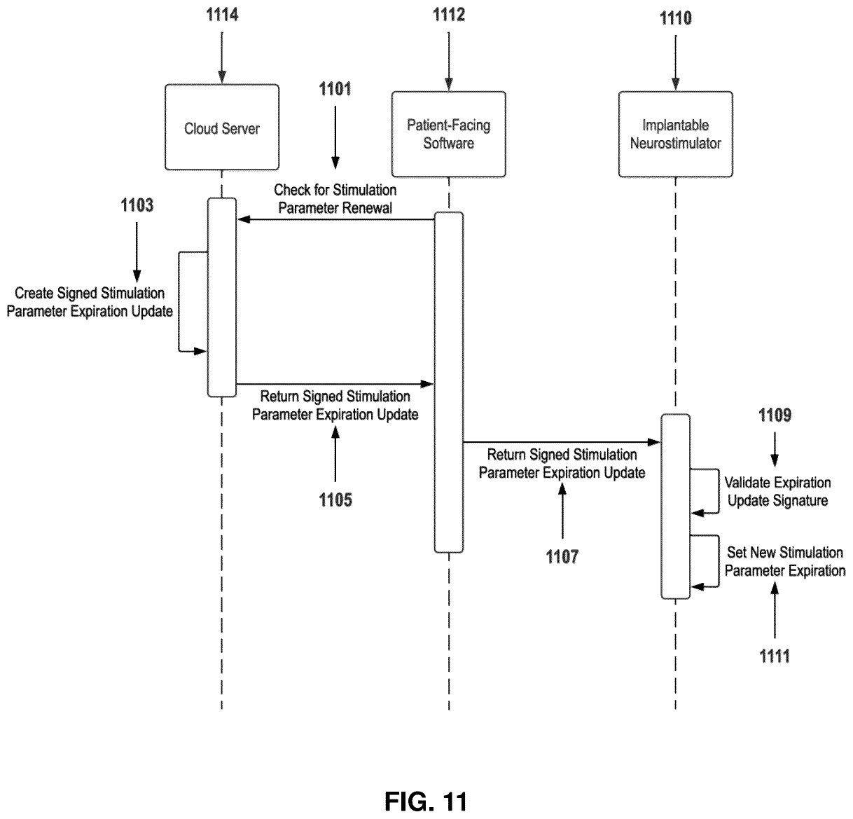

FIG. 11 is a sequence diagram that describes a prescription refill validation sequence for a therapy system as discussed herein, including a therapy system including an implant 1110, a patient-facing software application 1112, and a cloud server 1114.

At step 1101 of the sequence, patient can use a patient-facing software application 1112 to request a digital prescription refill for their therapy system. The request for refill can be handled by the cloud server 1114 which may validate the credentials of the patient.

At step 1103, cloud server creates a signed stimulation parameter update that may include a new expiration date for the digital prescription, a new set of maximum doses, or other adjustments to the neurostimulation parameters that the physician sees fit. Creation of this digital certificate is only possible with possession of one or more secret keys. The secret key(s) may, alternatively, be stored on more private and guarded servers, or certified hardware security modules. The time that is returned in the digital certificate package will contain the current date and time according to the cloud server.

At step 1105, the cloud server can return a stimulation parameter update certificate, that is digitally signed with a secret key, to the patient-facing software system. This stimulation parameter update is returned to the implant in step 1107. Although examples described herein discuss this communication as being between the patient-facing software and the implant, in other examples it should be understood that the patient-facing software can communicate with other components of the therapy system as intermediary devices, including an external charger.

At step 1109, the implantable neurostimulator, which comes preprogrammed with a corresponding public key to the private key from the private server (asymmetric encryption) or a shared secret key (symmetric encryption), can validate the stimulation parameter update/refill certificate from the patient-facing application. If the update/refill cannot be validated, the implant can be configured to block the update/refill. At step 1111, the digital prescription of the implant can be updated if the refill was validated in step 1109.

Many techniques can be used to provide prescription expiration/refills in a secure manner. In one example, asymmetric cryptography is used in which public keys can be added to the patient-facing application so that prescription refills can be authenticated. Refill tokens can then be generated on the server-side with physician-specific accounts. The tokens can be signed with a private key and authenticated with the public key. In some examples, the tokens are unique to a patient's individual prescription based on a unique identifier on the patient's implanted neurostimulator and the prescription schedule details including the date and time of scheduled doses. Refill tokens can also be cached on the patient's external charger to be delivered to the neurostimulator upon charging. Refills can be cryptographically guaranteed to work on specific implants and time-stamped prescriptions. The date and time of the prescription can be updated to the token upon refill so that a refill token cannot be reused.

The software infrastructure between the patient-facing application, the implanted neurostimulator, the external charger, the cloud, and physician accounts/devices may require full stack development to ensure secure prescriptions and prescription refills. In one example, both the implanted neurostimulator's bootloader and the companion application require asymmetric cryptography support. To provide additional security, the bootloader may only allow code-signed applications, and the public key must be stored in code-signed application code. The implanted microstimulator may be programmed to allow refill updates to be applied, and the energizer may be programmed to allow refill updates to be cached. As described above, a secure, cloud-based private key may be used to generate tokens, and only physician accounts/devices can be used to authorize refills.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and examples such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific examples have been illustrated and described herein, any arrangement calculated to achieve the same purpose may be substituted for the specific examples shown. This disclosure is intended to cover any and all adaptations or examples of various examples. Combinations of the above examples, and other examples not specifically described herein, will be apparent to those of skill in the art upon reviewing the above description.

What is claimed is:

1. A method of setting parameters of an implanted therapy system, the method comprising:
receiving, in the implanted therapy system, a set of neural stimulation parameters from a user with a digitally signed certificate, wherein the digitally signed certificate was provided to the user by a remote server after verifying that the user is a clinician who has provided a recognized user name and password to the remote server;
verifying, by the implanted therapy system, that a digital signature on the digitally signed certificate corresponds to an authorized user by either:
(1) using a unique, implant-specific key to confirm the digital signature, wherein the implant-specific key is known only to the implanted therapy system and to the remote server, or
(2) using a public key maintained in the implanted therapy system to confirm the digital signature corresponds to a private key held by only the remote server; and
setting neural stimulation parameters of the implanted therapy system based on the set of neural stimulation parameters received from the authorized user.

2. The method of claim 1, wherein the neural stimulation parameters affect a neural stimulation of a Cholinergic Anti-inflammatory Pathway (NCAP).

3. The method of claim 2, wherein the neural stimulation of the Cholinergic Anti-inflammatory Pathway (NCAP) provides therapeutic intervention for inflammation-mediated diseases or disorders.

4. The method of claim 2, wherein the neural stimulation of the Cholinergic Anti-inflammatory Pathway (NCAP) provides therapeutic intervention for rheumatologic diseases or disorders.

5. The method of claim 1, wherein the neural stimulation parameters comprise an expiration date and/or time for the neural stimulation parameters.

6. The method of claim 5, further comprising suspending neural stimulation by the implanted therapy system after the expiration date and/or time or after a specified number of stimulation treatments have been delivered as counted in a nonvolatile memory of the implanted therapy system.

7. The method of claim 5, further comprising renewing the expiration date and/or time by receiving a second communication from the user that is verified by the implanted therapy system by verifying a digital signature on a second digitally signed certificate received with the second communication including the renewed expiration date and/or time.

8. The method of claim 1, further comprising verifying a date and/or time of the implanted therapy system using a set of neural stimulation parameters from a second digitally signed certificate and verifying, by the implanted therapy system, that a digital signature on the second digitally signed certificate corresponds to an authorized user and setting neural stimulation parameters of the implanted therapy system based on the set of neural stimulation parameters received from the authorized user once the digital signature on the second digitally signed certificate is verified.

9. A method of setting a date and/or time of an implanted therapy system, the method comprising:
receiving, in the implanted therapy system, a date and/or time setting for the implanted therapy system with a digitally signed certificate, wherein the digitally signed certificate was provided by a remote server;
verifying, by the implanted therapy system, that a digital signature on the digitally signed certificate corresponds to an authorized instruction by either:
(1) using a unique, implant-specific key to confirm the digital signature, wherein the implant-specific key is known only to the implanted therapy system and to the remote server, or
(2) using a public key maintained in the implanted therapy system to confirm the digital signature corresponds to a private key held by only the remote server; and
modifying the date and/or time setting of the implanted therapy system based on the received date and/or time setting once the digital signature is verified.

10. The method of claim 9, further comprising modifying operation of the implanted therapy system based on a communication received from the authorized instruction.

11. The method of claim 10, wherein modifying operation of the implanted therapy system comprises setting electrical stimulation parameters for the implantable therapy system.

12. The method of claim 11, wherein the electrical stimulation parameters include an expiration date for the electrical stimulation parameters.

13. The method of claim 12, further comprising suspending electrical stimulation by the implanted therapy system after the expiration date or after a specified number of stimulation treatments have been delivered as counted in a nonvolatile memory of the implanted therapy system.

14. The method of claim 12, further comprising renewing the expiration date and/or time by receiving a second communication from a user that is verified by the implanted therapy system by verifying a digital signature on a second digitally signed certificate received with the second communication including the renewed expiration date and/or time.

15. The method of claim 9, further comprising verifying a date and/or time of the implanted therapy system by receiving, in the implanted therapy system, a date and/or time setting for the implanted therapy system with a second digitally signed certificate, verifying that a digital signature on the second digitally signed certificate corresponds to an authorized date and/or time setting, and modifying the date and/or time setting of the implanted therapy system based on the received date and/or time setting once the digital signature on the second digitally signed certificate is verified.

16. A method of updating software and/or firmware of an implanted therapy system, the method comprising:
receiving, in the implanted therapy system, a software and/or firmware update for the implanted therapy system with a digitally signed certificate, wherein the digitally signed certificate was provided by a remote server;
verifying, by the implanted therapy system, that a digital signature on the digitally signed certificate corresponds to an authorized instruction by either:
(1) using a unique, implant-specific key to confirm the digital signature, wherein the implant-specific key is known only to the implanted therapy system and to the remote server, or
(2) using a public key maintained in the implanted therapy system to confirm the digital signature corresponds to a private key held by only the remote server; and
updating the software and/or firmware of the implanted therapy system based on the received software and/or firmware update once the digital signature is verified.

17. The method of claim 16, wherein updating the software and/or firmware of the implanted therapy system comprises updating or modifying an existing prescription.

18. The method of claim 16, wherein updating the software and/or firmware of the implanted therapy system comprises adding or modifying electrical stimulation parameters for the implanted therapy system.

19. The method of claim 16, wherein updating the software and/or firmware of the implanted therapy system comprises adding or modifying neural stimulation parameters for the implanted therapy system.

20. The method of claim 16, further comprising verifying a date and/or time of the implanted therapy system by:
receiving, in the implanted therapy system, a software and/or firmware update for the implanted therapy system from a second digitally signed certificate;
verifying, by the implanted therapy system, that a digital signature on the second digitally signed certificate corresponds to an authorized user by either:
(1) using a unique, implant-specific key to confirm the digital signature, wherein the implant-specific key is known only to the implanted therapy system and to the remote server, or
(2) using a public key maintained in the implanted therapy system to confirm the digital signature corresponds to a private key held by only the remote server; and
updating the software and/or firmware of the implanted therapy system based on the received software and/or firmware update once the digital signature on the second digitally signed certificate is verified.
